(19) United States
(12) Patent Application Publication
(10) Pub. No.: US 2024/0366138 A1
(43) Pub. Date: Nov. 7, 2024

(54) EARLY DETECTION OF A HEART ATTACK BASED ON ELECTROCARDIOGRAPHY AND CLINICAL SYMPTOMS

(71) Applicants: Seyyed Abbas Atyabi, Tehran (IR); Mohammad Ali Niknami, Tehran (IR); Maryam Niknami, Tehran (IR); Afsaneh Maleki, Tehran (IR); Payam Niknami, Tehran (IR); Parnia Niknami, Tehran (IR); Elham Eshraghi, Vancouver (CA); Reyhane Rahimpour, Tehran (IR)

(72) Inventors: Seyyed Abbas Atyabi, Tehran (IR); Mohammad Ali Niknami, Tehran (IR); Maryam Niknami, Tehran (IR); Afsaneh Maleki, Tehran (IR); Payam Niknami, Tehran (IR); Parnia Niknami, Tehran (IR); Elham Eshraghi, Vancouver (CA); Reyhane Rahimpour, Tehran (IR)

(21) Appl. No.: 18/703,727

(22) PCT Filed: Oct. 24, 2022
(86) PCT No.: PCT/CA2022/051568

§ 371 Date: Apr. 23, 2024

Related U.S. Application Data
(60) Provisional application No. 63/271,193, filed on Oct. 24, 2021.

(51) Int. Cl. A61B 5/349; A61B 5/0205; A61B 5/355; A61B 5/358; A61B 5/363; A61B 5/366; G06N 5/048

(52) U.S. Cl. CPC A61B 5/349; A61B 5/0205; A61B 5/355; A61B 5/358; A61B 5/363; A61B 5/366; G06N 5/048

(57) ABSTRACT

A method for early detection of a heart attack in a subject. The method includes acquiring a plurality of clinical symptoms from the subject, acquiring a gender of the subject, acquiring an age of the subject, acquiring a raw ECG signal from the subject, generating an averaged ECG signal from the raw ECG signal, acquiring a plurality of ECG features from the averaged ECG signal, designing a fuzzy inference system based on a set of rules associated with the plurality of clinical symptoms, the gender, the age, and the plurality of ECG features, and determining an occurrence of the heart attack utilizing the fuzzy inference system.

### CROSS-REFERENCE TO RELATED APPLICATIONS

This application is a national stage of PCT International Application No. PCT/CA2022/051568, filed Oct. 24, 2022, which claims the benefit of priority from U.S. Provisional Application No. 63/271,193, filed on Oct. 24, 2021, the entire contents of each of which are incorporated herein by reference.

### TECHNICAL FIELD

The present disclosure generally relates to signal processing, and particularly, to biomedical signal processing.

### BACKGROUND OF THE INVENTION

Acute myocardial infarction (AMI) known as the most common form of coronary artery diseases is a major cause of morbidity and mortality worldwide. Every year, millions of people around the world die from MI or simply from heart attack. The main cause of heart attack is the blockage of blood flow to the heart muscles, caused by a damage or disease in the heart's major blood vessels (coronary arteries).

The highest risk of fatality occurs within the initial hours of onset of AMI. Thus early and rapid diagnosis of AMI is crucial for the timely initiation of evidence-based treatment leading to the better prognosis. Improper diagnosis of patients with chest pain often leads to inappropriate admission of patients without AMI and vice versa. In other words, delays in assessment of AMI especially in those patients with chest-pain dramatically increase the mortality and morbidity rates, while ruling out of AMI leads to save their lives and also profound effects on the patient management in the emergency department (ED).

According to the report of Center for Disease Control and Prevention in the United States, heart disease is the leading cause of death for both men and women. About 655,000 people die of heart disease in the United States every year—that's 1 in every 4 deaths. In the United States, someone has a heart attack every 40 seconds. Every year, about 805,000 Americans have a heart attack. Of these, 605,000 are a first heart attack, 200,000 happen to people who have already had a heart attack, and about 1 in 5 heart attacks is silent—the damage is done, but the person is not aware of it.

According to the latest clinical cardiology guidelines, the three main bases of the early diagnosis of MI or heart attack in the ED are clinical evaluation, 12-lead standard ECG interpretation, and measurement of the level of Troponin-T or Troponin-I proteins in the blood. These proteins are released when the heart muscle is damaged, such as in the case of heart attacks or even myocardial ischemia. The more damages or necrosis cites in the heart, the greater amount of Troponin-T and Troponin-I is observed in the blood samples.

Electrocardiography (ECG) has been a major cardiovascular diagnostic tool. Taking into account that ECG unit is simple, small, mobile, universally available and inexpensive, the standard 12-lead ECG has still remained a particularly attractive cardiovascular diagnostic tool for many years. The particular importance of the standard 12-lead ECG is its pivotal role in identifying patients with complete occlusions of large epicardial vessels. A complete vascular occlusion results in transmural ischemia, the maximal grade of ischemia, which is reflected by ST-elevation. This subgroup of patients with so-called ST-elevation myocardial infarction (STEMI) is at highest risk for adverse events and needs immediate reperfusion therapy such as percutaneous transluminal coronary angioplasty (PTCA), coronary artery bypass grafting (CABG) or medical therapy. However, only a minority of acute MI patients presents with transmural ischemia and ST-elevation, whereas the majority of AMI patients has only nontransmural ischemia, which result in either ST-depression, Twave inversion or no ECG change at all. These groups of AMI patients with nontransmural ischemia and without ST-elevation are clinically known as non-ST-elevation myocardial infarction (NSTEMI) patients.

In spite of the rapid progress in development of new hardware and software approaches for the standard 12-lead ECG detection, the cardiologists still mainly rely on the direct visual assessment of the standard 12-lead ECG signals. Existing developments in ECG processing generally utilize ECG signals along with other physiological parameters to aid experts in interpreting ECG signal variations [U.S. Pat. Nos. 8,672,856B2 and 7,328,061B2]. Although recently major developments in early identification of stable angina and NSTEMI patients were reported by proposing more accurate cardiac troponin assays, the progress in the analysis and interpretation of the 12-lead ECG was very limited over the years, and the applied conventional criteria for ECG interpretation have remained fundamentally unchanged for more than 25 years. The available diagnostic criteria for these patients are mostly focused on ST-depression and T-wave inversion. On the basis of these ECG criteria, at least 25% of patients with acute MI present with no diagnostic ECG abnormalities.

Nowadays, denying the diagnostic reliability of ECG interpretation by cardiology community and focusing more and more on the available alternative time-consuming assessment techniques (such as troponin testing) to diagnose the occurrence of MI, have resulted in losing the golden time of treatment for MI patients and exposing them to the risk of further dangerous side effects of their disease or even death. It means that, despite the massive studies in this field so far, as the best-case scenario and according to the recent developed assays and introduced clinical guidelines, the occurrence of AMI can be diagnosed at least between one to three hours after ED attendance. Although the current achievements are very valuable for MI patients, there is still a considerable clinical need of shortening the current diagnosis time to a definitive timely assessment. There is, therefore, a need for a method for early detection of heart attack that may provide a fast and a cost-efficient means for diagnosis of ECG and clinical symptoms.

### SUMMARY OF THE INVENTION

This summary is intended to provide an overview of the subject matter of the present disclosure, and is not intended to identify essential elements or key elements of the subject matter, nor is it intended to be used to determine the scope of the claimed implementations. The proper scope of the present disclosure may be ascertained from the claims set forth below in view of the detailed description below and the drawings.

In one general aspect, the present disclosure describes an exemplary method for early detection of a heart attack in a subject. An exemplary method may include acquiring a plurality of clinical symptoms from the subject, acquiring a gender of the subject, acquiring an age of the subject, acquiring a raw electrocardiography (ECG) signal from the subject at a diagnosis time period, generating a denoised ECG signal by applying a first wavelet transform on the raw ECG signal, generating an artifact-free ECG signal by applying a second wavelet transform on the denoised ECG signal, generating a filtered ECG signal by applying a finite impulse response (FIR) filter on the artifact-free ECG signal, extracting an averaged ECG signal of a plurality of averaged ECG signals from the filtered ECG signal, acquiring a plurality of ECG features from the averaged ECG signal and the filtered ECG signal, generating a plurality of clinical symptoms fuzzy sets associated with the plurality of clinical symptoms, generating a plurality of gender-age fuzzy sets associated with the gender and the age, generating a plurality of ECG fuzzy sets associated with the plurality of ECG features, generating a myocardial infarction (MI) class corresponding to occurrence of an MI in the subject and a non-MI class corresponding to an absence of MI in the subject, designing a fuzzy inference system based on a set of rules, mapping each of the plurality of clinical symptoms, the gender, the age, and the plurality of ECG features to a respective fuzzy input of a plurality of fuzzy inputs, and determining an occurrence of the heart attack in the subject by applying the plurality of fuzzy inputs to the fuzzy inference system.

An exemplary gender may include a male or a female. An exemplary averaged ECG signal may include a QRS complex, an ST segment, and a T wave. Each exemplary rule of the set of rules may include mapping a respective combination of a respective clinical symptoms fuzzy set of the plurality of clinical symptoms fuzzy sets, a respective gender-age fuzzy set of the plurality of gender-age fuzzy sets, and a respective ECG fuzzy set of the plurality of ECG fuzzy sets to one of the MI class or the non-MI class.

Other exemplary systems, methods, features and advantages of the implementations will be, or will become, apparent to one of ordinary skill in the art upon examination of the following figures and detailed description. It is intended that all such additional systems, methods, features and advantages be included within this description and this summary, be within the scope of the implementations, and be protected by the claims herein.

### BRIEF DESCRIPTION OF DRAWINGS

The drawing figures depict one or more implementations in accord with the present teachings, by way of example only, not by way of limitation. In the figures, like reference numerals refer to the same or similar elements.

FIG. 1A shows a flowchart of a method for early detection of a heart attack in a subject, consistent with one or more exemplary embodiments of the present disclosure.

FIG. 1B shows a flowchart for acquiring a raw electrocardiography (ECG) signal from a patient, consistent with one or more exemplary embodiments of the present disclosure.

FIG. 1C shows a flowchart for detecting one of a depression or an elevation in an ST segment, consistent with one or more exemplary embodiments of the present disclosure.

FIG. 1D shows a flowchart for detecting a deformation in an ST segment, consistent with one or more exemplary embodiments of the present disclosure.

FIG. 1E shows a flowchart for detecting an abnormal morphology in a respective averaged ECG signal of a plurality of averaged ECG signals, consistent with one or more exemplary embodiments of the present disclosure.

FIG. 2A shows a schematic of a system for early detection of a heart attack in a subject, consistent with one or more exemplary embodiments of the present disclosure.

FIG. 2B shows a schematic of a fuzzy inference system (FIS), consistent with one or more exemplary embodiments of the present disclosure.

FIG. 3A shows a raw ECG signal, consistent with one or more exemplary embodiments of the present disclosure.

FIG. 3B shows a denoised ECG signal, consistent with one or more exemplary embodiments of the present disclosure.

FIG. 3C shows an artifact-free ECG signal, consistent with one or more exemplary embodiments of the present disclosure.

FIG. 3D shows a filtered ECG signal, consistent with one or more exemplary embodiments of the present disclosure.

FIG. 3E shows an averaged ECG signal, consistent with one or more exemplary embodiments of the present disclosure.

FIG. 3F shows a plurality of averaged ECG signals that include a plurality of abnormal morphologies, consistent with one or more exemplary embodiments of the present disclosure.

FIG. 4 shows a diagram of a first membership function, consistent with one or more exemplary embodiments of the present disclosure.

FIG. 5 shows a high-level functional block diagram of a computer system, consistent with one or more exemplary embodiments of the present disclosure.

FIG. 6 shows different pages of a software application for early detection of heart attack implemented on a smart watch, consistent with one or more exemplary embodiments of the present disclosure.

### DETAILED DESCRIPTION OF THE INVENTION

In the following detailed description, numerous specific details are set forth by way of examples in order to provide a thorough understanding of the relevant teachings. However, it should be apparent that the present teachings may be practiced without such details. In other instances, well known methods, procedures, components, and/or circuitry have been described at a relatively high-level, without detail, in order to avoid unnecessarily obscuring aspects of the present teachings.

The following detailed description is presented to enable a person skilled in the art to make and use the methods and devices disclosed in exemplary embodiments of the present disclosure. For purposes of explanation, specific nomenclature is set forth to provide a thorough understanding of the present disclosure. However, it will be apparent to one skilled in the art that these specific details are not required to practice the disclosed exemplary embodiments. Descriptions of specific exemplary embodiments are provided only as representative examples. Various modifications to the exemplary implementations will be readily apparent to one skilled in the art, and the general principles defined herein may be applied to other implementations and applications without departing from the scope of the present disclosure. The present disclosure is not intended to be limited to the implementations shown, but is to be accorded the widest possible scope consistent with the principles and features disclosed herein.

Herein is disclosed an exemplary method for early detection of heart attack. An exemplary method may record an electrocardiography (ECG) signal from a subject via a single-lead ECG. A number of clinical symptoms, along with the gender and the age of the subject may also be obtained. An exemplary gathered data may be loaded to a fuzzy inference system (FIS) that is designed based on a set of rules that map different combinations of obtained data from patients to a determination of heart attack. An exemplary FIS may map the gathered data from the subject to a set of fuzzy inputs and may determine occurrence or absence of heart attack in the subject.

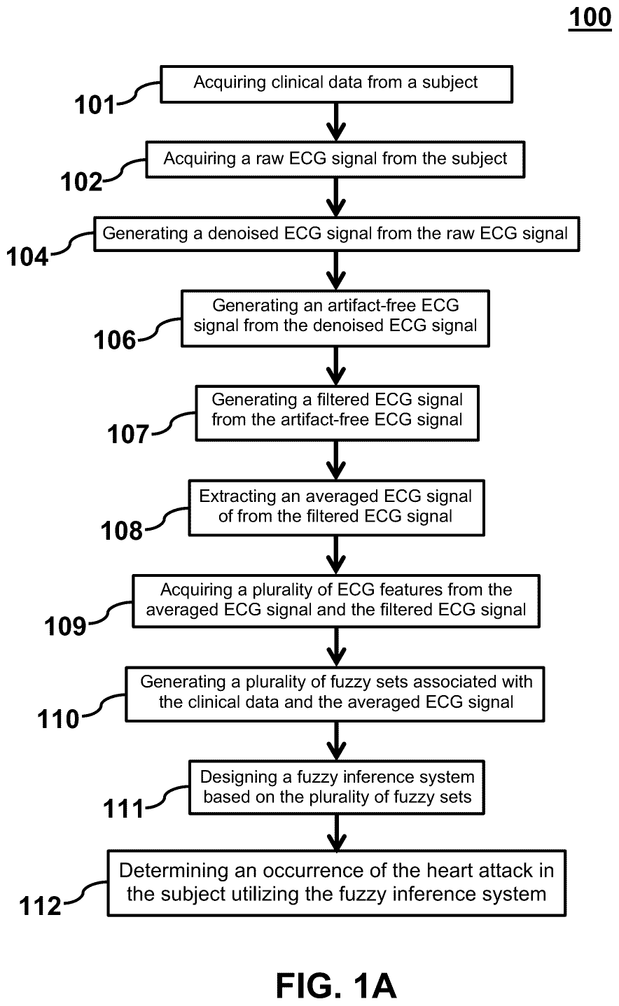

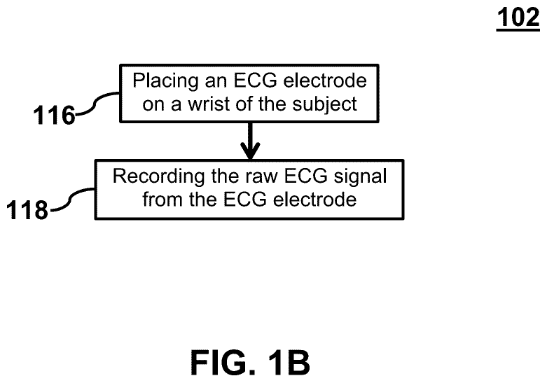

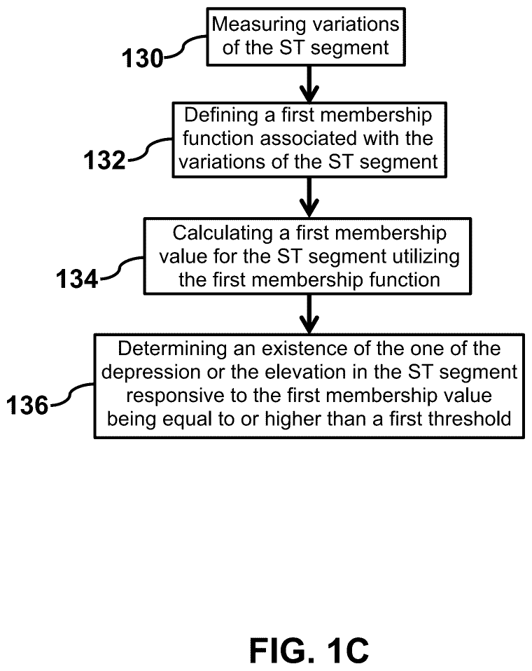

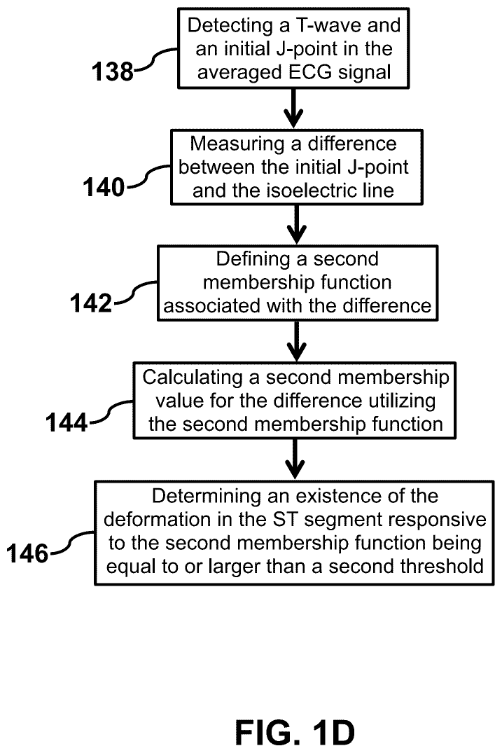

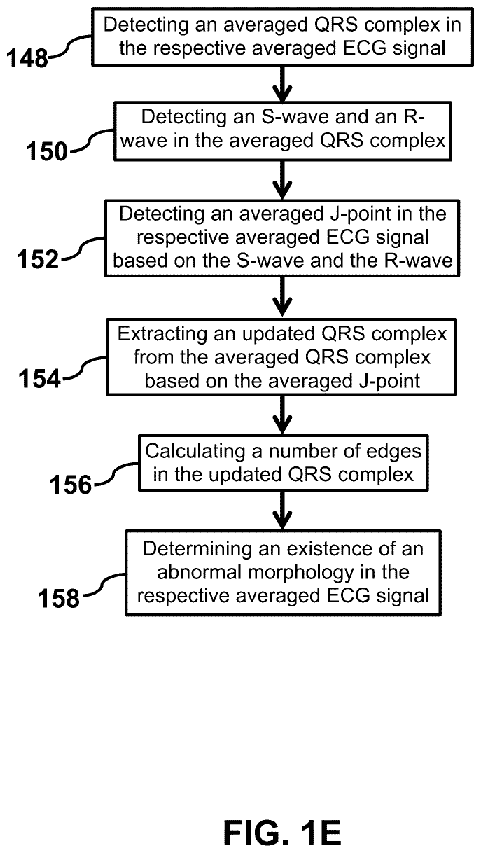

FIG. 1A shows a flowchart of a method for early detection of a heart attack in a subject, consistent with one or more exemplary embodiments of the present disclosure. An exemplary method 100 may include acquiring clinical data from the subject (step 101), acquiring a raw electrocardiography (ECG) signal from the (step 102), generating a denoised ECG signal by applying a first wavelet transform on the raw ECG signal (step 104), generating an artifact-free ECG signal by applying a second wavelet transform on the denoised ECG signal (step 106), generating a filtered ECG signal by applying a band-stop filter on the artifact-free ECG signal (step 107), extracting an averaged ECG signal of a plurality of averaged ECG signals from the filtered ECG signal (step 108), acquiring a plurality of ECG features from the averaged ECG signal and the filtered ECG signal (step 109), generating a plurality of fuzzy sets associated with the plurality of clinical data and the plurality of ECG features (step 110), designing a fuzzy inference system based on the plurality of fuzzy sets (step 111), and determining an occurrence of the heart attack in the subject utilizing the fuzzy inference system (step 112).

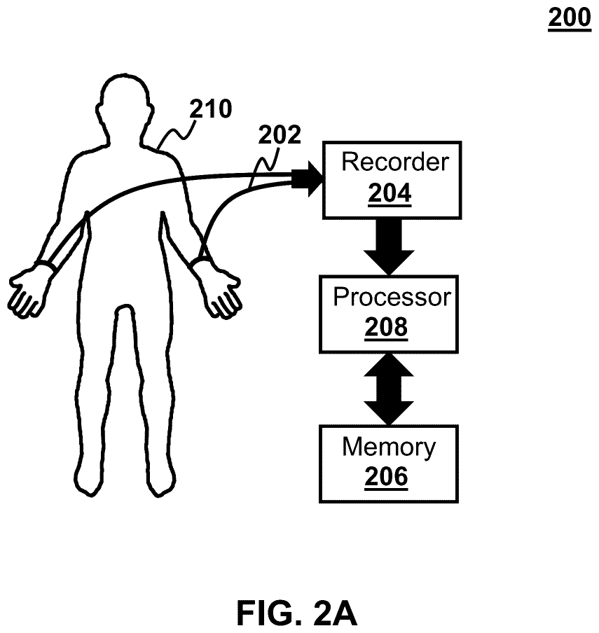

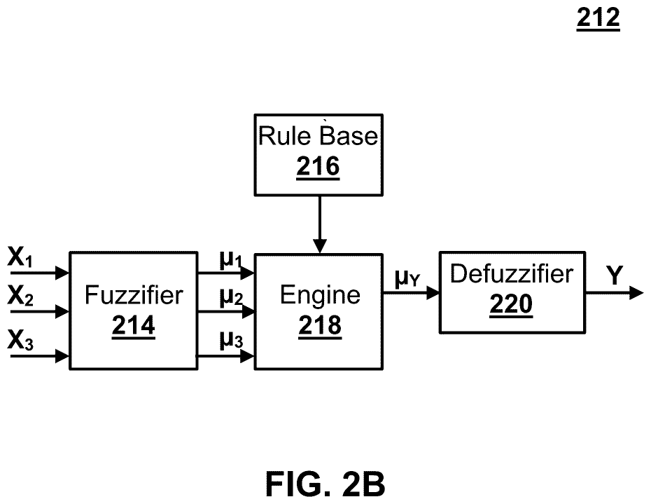

FIG. 2A shows a schematic of a system for early detection of a heart attack in a subject, consistent with one or more exemplary embodiments of the present disclosure. In an exemplary embodiment, different steps of method 100 may be implemented by utilizing an exemplary system 200. An exemplary system 200 may include an ECG electrode 202, an ECG recorder 204, a memory 206, and a processor 208. In an exemplary embodiment, ECG electrode 202 may be placed on a wrist of a subject 210.

In an exemplary embodiment, step 101 may include acquiring clinical data from subject 210. Exemplary clinical data may include a plurality of clinical symptoms, a gender (i.e., male or female) of subject 210, and an age of subject 210. In an exemplary embodiment, the plurality of clinical symptoms may include a first clinical symptom, a second clinical symptom, a third clinical symptom, a fourth clinical symptom, a fifth clinical symptom, and a sixth clinical symptom.

An exemplary first clinical symptom may include an on/off pain with a continuous duration of at least five minutes during a one hour period before a diagnosis time in at least one of a first plurality of regions having a total size larger than three times of a size of a fingertip of subject 210. In an exemplary embodiment, the first plurality of regions may include upper chest, middle chest (sternum), upper abdomen, neck, jaw, right shoulder, left shoulder, inside right arm, inside left arm, and between shoulders in back. An exemplary on/off pain may pause about 10-15 minutes before starting again. In an exemplary embodiment, a “diagnosis time” may refer to a time at which method 100 may be implemented.

An exemplary second clinical symptom may include at least one of fainting or both the following symptoms: First, at least one of shortness of breath, light headedness, diabetes, and hypertension. Second, at least one of sweating and on/off pain with a continuous duration of at least five minutes.

In an exemplary embodiment, the first clinical symptom and the second clinical symptom may be referred to as “typical myocardial infarction (MI) symptoms” since they may be strongly important from a medical expert's viewpoint for early diagnosis of a heart attack.

In an exemplary embodiment, the third clinical symptom and the fourth clinical symptom may include symptoms similar to the first clinical symptom and the second clinical symptom. However, in an exemplary embodiment, the third clinical symptom and the fourth clinical symptom may have been felt by subject 210 then disappeared from about 24 hours until about one hour before the diagnosis time. In an exemplary embodiment, the third clinical symptom and the fourth clinical symptom may be referred to as “high risk for MI symptoms.”

In an exemplary embodiment, the fifth clinical symptom may include an atypical MI pain during a one hour period before the diagnosis time in at least one of a second plurality of regions. An exemplary second plurality of regions may include upper chest, middle chest (sternum), upper abdomen, neck, jaw, right shoulder, left shoulder, inside right arm, inside left arm, and between shoulders in back. In an exemplary embodiment, the atypical MI pain may be either continuous (i.e., not an on/off pain) or in an area that is smaller than a size of the fingertip of subject 210. In an exemplary embodiment, the fifth clinical symptom may be referred to as an “atypical MI symptom” since it is not a typical symptom for the diagnosis of heart attack alone and may be representing other diseases.

In an exemplary embodiment, the sixth clinical symptom may include any symptom (or no symptom at all) that may be different from each of the first clinical symptom, the second clinical symptom, the third clinical symptom, the fourth clinical symptom, and the fifth clinical symptom. In an exemplary embodiment, the sixth clinical symptom may describe that subject 210 feels no chest pain or discomfort. Also, an exemplary sixth clinical symptom may show that subject 210 does not have any symptoms that may be associated with MI.

In further detail with respect to step 102, FIG. 1B shows a flowchart for acquiring a raw ECG signal from a patient, consistent with one or more exemplary embodiments of the present disclosure. In an exemplary embodiment, acquiring the raw ECG signal (step 102) may include placing an ECG electrode on a wrist of the subject (step 116) and recording the raw ECG signal from the ECG lead (step 118).

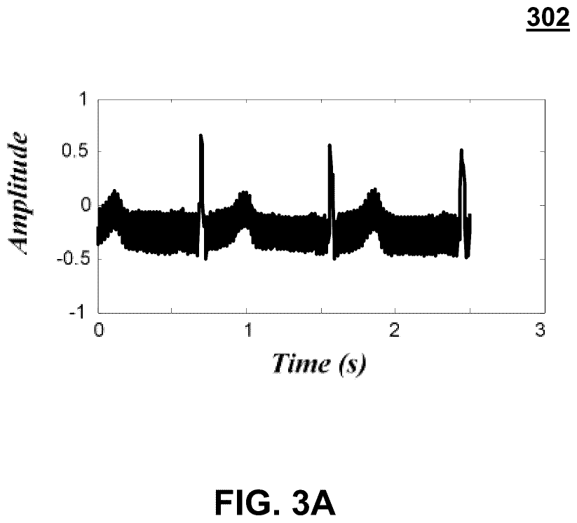

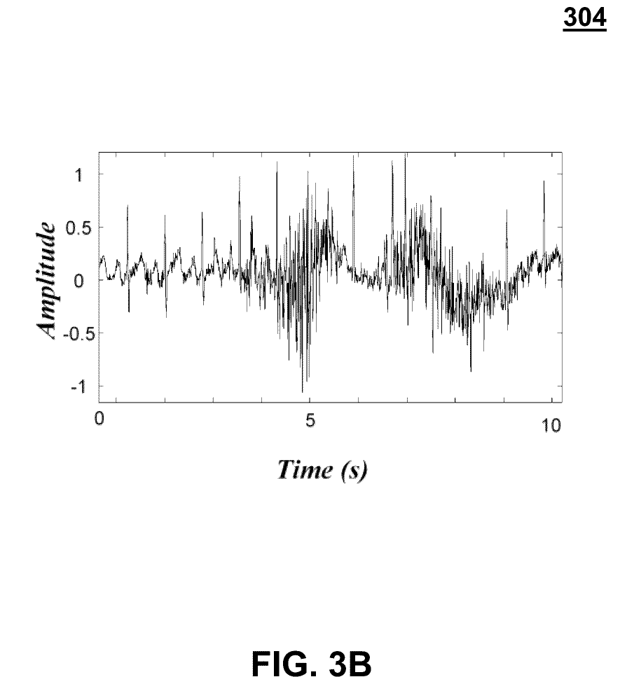

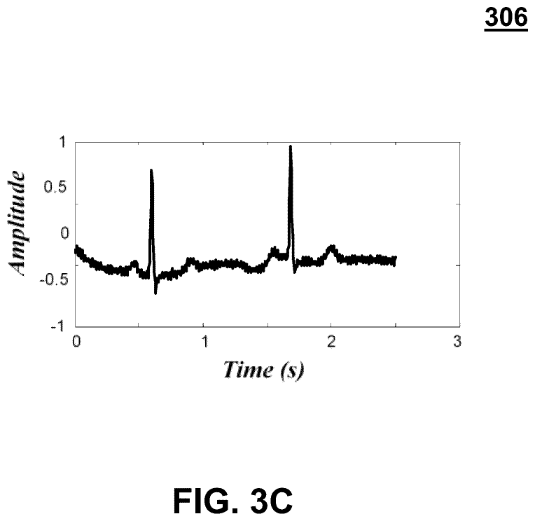

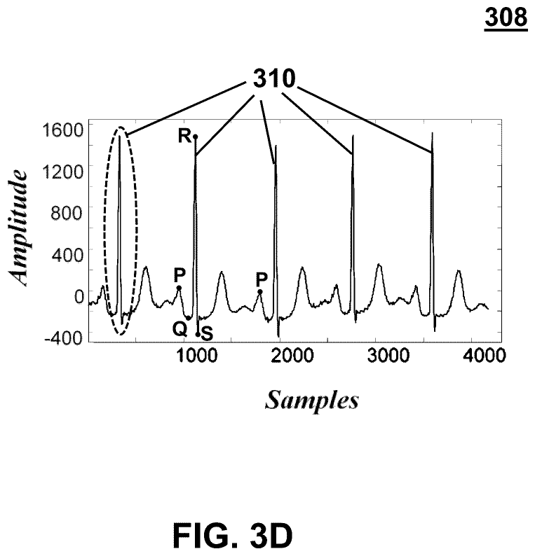

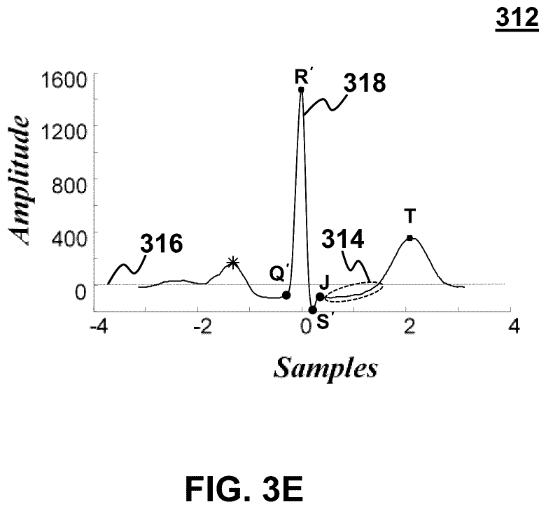

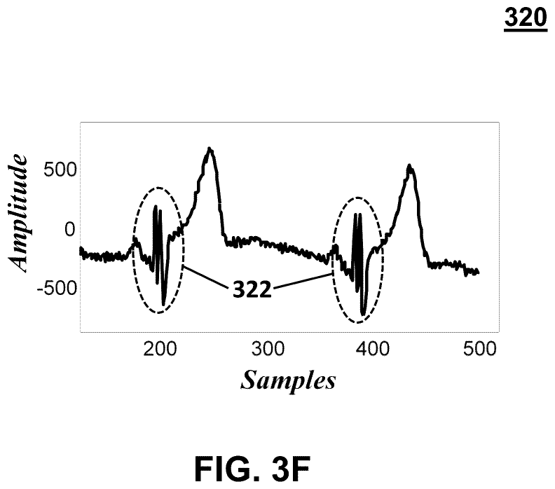

For further detail regarding step 116, in an exemplary embodiment, ECG electrode 202 may be embedded in an electronic gadget such as a smart watch, a smart wristband, etc. In further detail with regards to step 118, FIG. 3A shows a raw ECG signal, consistent with one or more exemplary embodiments of the present disclosure. In an exemplary embodiment, ECG recorder 204 may be utilized for recording a raw ECG signal 302. In an exemplary embodiment, ECG recorder 204 may send raw ECG signal 302 to processor 208 to process raw ECG signal 302 according to steps 104-110 of method 100. In an exemplary embodiment, different steps of method 100 may be stored in memory 206 to be accessed and executed by processor 208.

Referring again to FIG. 1A, in an exemplary embodiment, step 104 may include generating a denoised ECG signal by applying a first wavelet transform on raw ECG signal 302. FIG. 3B shows a denoised ECG signal, consistent with one or more exemplary embodiments of the present disclosure. In an exemplary embodiment, the first wavelet transform may include a discrete wavelet transform. In an exemplary embodiment, noises of wavelet coefficients may be estimated by applying a discrete wavelet transform at on raw ECG signal 302. Then, by defining an appropriate threshold level, the noises may be removed and an exemplary denoised ECG signal 304 may be obtained.

In an exemplary embodiment, step 106 may include generating an artifact-free ECG signal by applying a second wavelet transform on denoised ECG signal 304. FIG. 3C shows an artifact-free ECG signal, consistent with one or more exemplary embodiments of the present disclosure. In an exemplary embodiment, the second wavelet transform may include a discrete wavelet transform. In an exemplary embodiment, artifact locations may be estimated by applying a discrete wavelet transform on denoised ECG signal 304. Exemplary artifacts may be a result of subject's motion or contacting with vibrating or electrical tools. In an exemplary embodiment, a sliding window may be moved on denoised ECG signal 304 and a discrete wavelet transform may be applied on a segment of denoised ECG signal 304 that is inside the sliding window. Then, noises of wavelet coefficients may be estimated. Next, by comparing obtained noise level with a proper threshold, the quality of the segment inside the sliding window is evaluated. If the noise level is above the threshold, an artifact may be identified within the segment. In an exemplary embodiment, this process may continue until the end of denoised ECG signal 304 and artifact locations may be identified throughout denoised ECG signal 304. Then, by selecting segments without any identified artifact, an exemplary artifact-free ECG signal 306 may be obtained.

In an exemplary embodiment, step 107 may include generating a filtered ECG signal by applying an FIR filter on artifact-free ECG signal 306. FIG. 3D shows a filtered ECG signal, consistent with one or more exemplary embodiments of the present disclosure. In an exemplary embodiment, the FIR filter may include a band-stop filter that may be applied on artifact-free ECG signal 306 to remove certain frequency related noises, such as power line noises in a range of bout 48-51 Hz. In an exemplary embodiment, the FIR filter may further include a high pass filter that may be applied on artifact-free ECG signal 306 to eliminate low-frequency noises. By applying the FIR filter on artifact-free ECG signal 306, an exemplary filtered ECG signal 308 may be obtained. In an exemplary embodiment, filtered ECG signal 308 may include a plurality of QRS complexes 310. An exemplary QRS complex of plurality of QRS complexes 310 may include Q, R, and S edges.

In an exemplary embodiment, step 108 may include extracting an averaged ECG signal of from filtered ECG signal 308. FIG. 3E shows an averaged ECG signal, consistent with one or more exemplary embodiments of the present disclosure. Extracting an exemplary averaged ECG signal 312 may include extracting three consecutive QRS complexes of plurality of QRS complexes 310 from filtered ECG signal 308. In an exemplary embodiment, each of the three consecutive QRS complexes may be delineated by applying a stationary wavelet transform (SWT, also known as a'trous discrete wavelet transform) on filtered ECG signal 308. Each exemplary QRS complex may correspond to a heartbeat pulse that may be located between onsets of two exemplary consecutive P-waves in filtered ECG signal 308. In an exemplary embodiment, a respective onset of each P-wave may be detected in filtered ECG signal 308 similar to delineating an associated QRS complex. Next, three exemplary consecutive heartbeat pulses (each being located between two successive P-wave onsets) may be extracted from filtered ECG signal 308 and averaged to obtain an exemplary averaged ECG signal 310. As a result, in an exemplary embodiment, remaining noises in filtered ECG signal 308 may be considerably removed in averaged ECG signal 312. Referring to FIGS. 3D and 3E, in an exemplary embodiment, averaged ECG signal 312 may be smoother than filtered ECG signal 308.

Referring again to FIG. 1A, in an exemplary embodiment, step 109 may include acquiring a plurality of ECG features from averaged ECG signal 312 and the filtered ECG signal 308 by assessment of a first plurality of features in averaged ECG signal 312 and assessment of a second plurality of features in one of averaged ECG signal 312 or filtered ECG signal 308.

In an exemplary embodiment, the first plurality of features may include an elevation or a depression in the ST segment, a pathologic Q-wave or an abnormal morphology in the QRS complex, and the T wave being a tall T wave. In an exemplary embodiment, the first plurality of features may be referred to as “in favor of MI ECG features” since they may describe conditions that may be important from a medical expert's viewpoint for discriminating between subjects at MI (risk of heart attack) and non-MI ones.

In an exemplary embodiment, the second plurality of features may include a deformation in the ST segment, a severe bradycardia in filtered ECG signal 308, and the T wave being an inverted T wave, a tented T wave, a flat T wave, or a biphasic T wave. In an exemplary embodiment, the second plurality of features may be referred to as “suspect of MI ECG features” since they may describe suspicions conditions from a medical expert's viewpoint for the occurrence of an onset of an MI or a soon-to-be happening heart attack.

Severe bradycardia is detected when the heart rate is lower than 40 beats per minute. Heart rate can be measured from different formulas, however since the heart rate is supposed to be calculated for severe bradycardia detection, the following formula is recommended which is effective even in case of irregular rhythms:

$$Heart Rate = Number of QRS complexes over a 6 - second interval multiplied by 10$$

In an exemplary embodiment, a 6-second sliding window may be used on a pre-delineated ECG, and in each window, the number of QRS complexes may be counted and then multiplied by 10. When the sliding window goes to the end of the delineated ECG, the median of the calculated vector of heart rates is selected as the median heart rate over the total period of the delineated ECG. If the median heart rate is lower than 40 beats per minute, then the occurrence of severe bradycardia is detected.

In an exemplary embodiment, the QRS complex may include a pathologic Q wave if the following condition is satisfied:

$$0.25 {A}_{R} \leq {A}_{{Q}_{ρ e a k}} AND \vert {Q}_{onset} - {Q}_{offset} \vert \geq 40 ms$$

where A_{R} is an amplitude of the R wave, A_{Q}_{ₚₑₐₖ} is an amplitude of the peak of the Q wave, Qₒₙₛₑₜ is an onset time of the Q wave, and Q_{offset} is an offset time of the Q wave.

An exemplary “inverted T-wave” may refer to a T-wave whose normal upright shape is changed and becomes inverted. In an exemplary embodiment, a base of a “tented T wave” may quickly become narrow and are tented, as if pinched from above. In an exemplary embodiment, a “flat T wave” may refer to a T wave with an amplitude between about +0.1 mV to about −0.1 mV. In an exemplary embodiment, a “biphasic T wave” may refer to a T wave that swings up and then down and is inscribed on either side of the baseline.

In an exemplary embodiment, the delineated points of each T wave in each averaged ECG including the onset, peak, and offset points may be utilized to detect normal, tented, tall, biphasic, inverted, and flattened T waves according to the following:

$$T - wave is Normal if + 0.2 mV \leq {A}_{T \_ peak} \leq + 0.5 mV$$

$$T - wave is inverted if {A}_{{T}_{peak}} \leq - 0.2 mV$$

$$T - wave is Tall if + 0.5 mV \leq {A}_{{T}_{peak}} AND 150 ms \leq \vert {T}_{onset} - {T}_{offset} \vert$$

$$T - wave is Tented if + 0.5 mV \leq {A}_{{T}_{peak}} AND \vert {T}_{onset} - {T}_{offset} \vert \leq 150 ms$$

$$Flattened T - wave is detected if + 0.1 mV \leq {A}_{T \_ peak} \leq - 0.1 mV$$

where A_{T}_{ₚₑₐₖ} is an amplitude of the peak of the T wave, Tₒₙₛₑₜ is an onset time of the T wave, and T_{offset} is an offset time of the T wave.

Given the onset and offset of the T-wave, to detect the biphasic shape of a T wave, a number of extremum points of the T wave may be obtained and then the following condition is applied:

### T-Wave is Biphasic if

$${E}_{point} = 2 and + 0.2 \leq {A}_{{E}_{point 1}} AND {A}_{{E}_{point 2}} \leq - 0.2$$

where Eₚₒᵢₙₜ is a number of extremum points, A_{E}_{ₚₒᵢₙₜ₁} is an amplitude of a first extremum point, and A_{E}_{ₚₒᵢₙₜ₁} is an amplitude of a second extremum point.

FIG. 1C shows a flowchart for detecting one of a depression or an elevation in an ST segment, consistent with one or more exemplary embodiments of the present disclosure. Referring to FIGS. 1C and 3E, in an exemplary embodiment, an elevation or a depression in the ST segment in step 109 may include measuring variations of an ST segment 314 with respect to an isoelectric line 316 (step 130), defining a first membership function associated with the variations of the ST segment (step 132), calculating a first membership value for the ST segment utilizing the first membership function (step 134), and determining an existence of the one of the depression or the elevation in the ST segment responsive to the first membership value being equal to or higher than a first threshold (step 136). In an exemplary embodiment, ST segment 314 may refer to a segment in averaged ECG signal 312 between an averaged QRS complex 318 and a T-wave. In an exemplary embodiment, isoelectric line 316 may refer to a baseline of averaged ECG signal 312 where the signal has zero amplitude. In an exemplary embodiment, ST segment 314 and isoelectric line 316 may be detected by applying an SWT on averaged ECG signal 312, similar to the aforementioned delineation processes.

For further detail with respect to step 130, variations of an exemplary ST segment may include a depression or an elevation. In an exemplary embodiment, a depression may refer to a decrease of an ST segment's amplitude below an associated isoelectric line and an elevation may refer to an increase of an ST segment's amplitude above an associated isoelectric line. For example, referring to FIG. 3E, the variations of ST segment 314 include a depression since ST segment 314 lies below isoelectric line 316. In an exemplary embodiment, measuring ST segment 314 variations may include calculating an average of ST segment 314 variations with respect to isoelectric line 316.

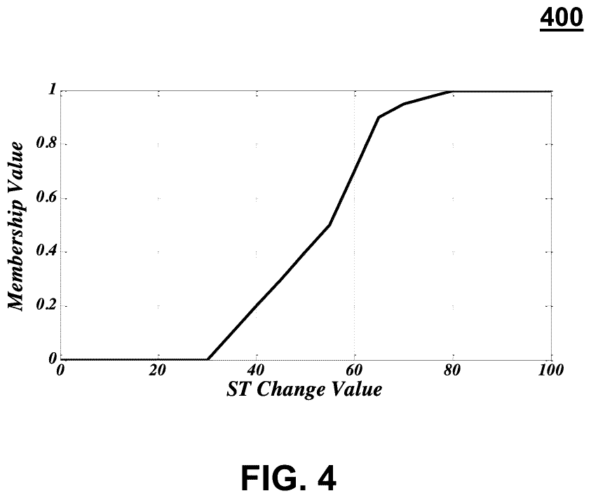

In further detail with regards to step 132, FIG. 4 shows a diagram of a first membership function, consistent with one or more exemplary embodiments of the present disclosure. An exemplary first membership function 400 may be utilized for mapping the variations of ST segment 314 to a number between 0 and 1. In an exemplary embodiment, first membership function 400 may be obtained empirically by applying different functions on ST segment 314.

For further detail with respect to step 134, calculating the first membership value may include applying a measured value of variations of ST segment 314 to first membership function 400 and extracting a corresponding output of first membership function 400 as the first membership value for ST segment 314.

In further detail regarding step 136, in an exemplary embodiment, the first membership value may be compared with a first threshold. An exemplary first threshold may be set equal to about 0.7 based on examining different threshold values. In an exemplary embodiment, if the first membership value is equal to or larger than the first threshold, an existence of a depression or an elevation may be determined in ST segment 314.

FIG. 1D shows a flowchart for detecting a deformation in an ST segment, consistent with one or more exemplary embodiments of the present disclosure. In an exemplary embodiment, step 124 may include detecting a T-wave and an initial J-point in averaged ECG signal 312 (step 138), measuring a difference between the initial J-point and the isoelectric line (step 140), defining a second membership function associated with the difference (step 142), calculating a second membership value for the difference utilizing the second membership function (step 144), and determining an existence of the deformation in the ST segment responsive to the second membership function being equal to or larger than a second threshold (step 146).

For further detail with respect to step 138, referring to FIG. 3E, an exemplary initial J-point may refer to an onset of ST segment 314. Therefore, in an exemplary embodiment, detecting the initial J-point may include extracting an onset J of ST segment 314. In an exemplary embodiment, detecting the T-wave may include detecting a (positive or negative) peak T of the T-wave by applying an SWT on averaged ECG signal 312, similar to detecting ST segment 314. In an exemplary embodiment, detecting the T-wave may include detecting a type of the T-wave. An exemplary type of the T-wave may include one of a normal T-wave and an inverted T-wave. In an exemplary embodiment, a normal T-wave may refer to a T-wave with a positive peak (i.e., a local maximum) and an inverted T-wave may refer to a T-wave with a negative peak (i.e., a local minimum). For example, the T-wave in averaged ECG signal 312 is a normal T-wave since it has a positive peak T.

In an exemplary embodiment, method 100 may further include determining a modified J-point on averaged ECG signal 312 responsive to detecting an inverted T-wave. An exemplary J-point modification may include modifying a location of the initial J-point on averaged ECG signal 312 by calculating a modified location Jₘ for the modified J-point according to an operation defined by the following:

$${J}_{m} = {J}_{i} - {f}_{s} / 5 \qquad Equation ( 1 )$$

where:
Jᵢ is a location of the initial J-point on averaged ECG signal 312, and
fₛ is a sampling frequency of raw ECG signal 302.

In an exemplary embodiment, Equation (1) may be empirically obtained by relocating onset J at different modified locations and examining the impact of different relocations on the performance of method 100. In an exemplary embodiment, method 100 may further include replacing the initial J-point with the modified J-point prior to measuring the difference between the initial J-point and isoelectric line 316 in step 140 according to Equation (1).

In further detail regarding step 140, in an exemplary embodiment, measuring the difference between the initial J-point and isoelectric line 316 may include calculating an absolute value of averaged ECG signal 312 amplitude at onset J due to a zero amplitude of averaged ECG signal 312 on isoelectric line 316.

In further detail with regards to step 142, an exemplary second membership function may be selected similar to or different from first membership function 400. An exemplary second membership function may be utilized for fuzzy decision making over the difference between the initial J-point and isoelectric line 316. An exemplary second membership function may be obtained empirically by applying different functions on averaged ECG signal 312.

For further detail with respect to step 144, calculating the second membership value may include applying a measured value of an exemplary difference between the initial J-point and isoelectric line 316 to the second membership function and extracting a corresponding output of the second membership function as the second membership value for ST segment 314.

For further detail regarding step 146, in an exemplary embodiment, the second membership value may be compared with a second threshold. An exemplary second threshold may be set equal to about 0.9 based on examining different threshold values. In an exemplary embodiment, if the second membership value is equal to or larger than the second threshold, an existence of a deformation may be determined in ST segment 314.

FIG. 1E shows a flowchart for detecting an abnormal morphology in a respective averaged ECG signal of a plurality of averaged ECG signals, consistent with one or more exemplary embodiments of the present disclosure. In an exemplary embodiment, detecting each of the plurality of abnormal morphologies may include detecting an averaged QRS complex in the respective averaged ECG signal (step 148), detecting an S-wave and an R-wave in the averaged QRS complex (step 150), detecting an averaged J-point in the respective averaged ECG signal based on the S-wave and the R-wave (step 152), extracting an updated QRS complex from the averaged QRS complex based on the averaged J-point (step 154), calculating a number of edges in the updated QRS complex (step 156), and determining an existence of an abnormal morphology of the plurality of abnormal morphologies in the respective averaged ECG signal responsive to a temporal duration of the updated QRS complex being less than a temporal threshold and the number of edges being larger than a lower limit (step 158).

For further detail with respect to step 148, in an exemplary embodiment, each of the plurality of averaged ECG signals may include an averaged QRS complex. For example, referring again to FIG. 3E, averaged ECG signal 312 may include averaged QRS complex 318. In an exemplary embodiment, averaged QRS complex 318 may be detected by applying an SWT on averaged ECG signal 312 similar to detecting plurality of QRS complexes 310 in filtered ECG signal 308.

In further detail regarding step 150, in an exemplary embodiment, averaged QRS complex 318 may include an R-wave and an S-wave. An exemplary R-wave may include an exemplary edge R′ and an exemplary S-wave may include an exemplary edge S′. Therefore, in an exemplary embodiment, each of the R-wave and an S-wave may be detected by detecting corresponding edges R′and S′, respectively.

For further detail with regards to step 152, in an exemplary embodiment, detecting the averaged J-point may include calculating a coefficient cff according to an operation defined by the following:

$$cff = \frac{{R}_{m} - Iso}{{S}_{m} - Iso} \qquad Equation ( 2 )$$

where:
Rₘ is a peak of the R-wave,
Sₘ is a peak of the S-wave, and
Iso is an amplitude of the isoelectric line.
   In an exemplary embodiment, Equation (2) may be empirically obtained for compensating the impact of different shapes of averaged QRS complex 318 on an accuracy of averaged J-point detection. In an exemplary embodiment, step 152 may further include setting a width W of a search range that may satisfy a set of conditions defined by the following:

$$0.4 {f}_{s} < W < 0.5 {f}_{s} , c f f \leq 0.1 \qquad Condition ( 1 a )$$

$$0.3 {f}_{s} < W < 0.4 {f}_{s} , 0.1 < c f f \leq 1.5 \qquad Condition ( 1 b )$$

$$0.1 {f}_{s} < W < 0.2 {f}_{s} , c f f > 1.5 \qquad Condition ( 1 c )$$

According to Condition (1a), in an exemplary embodiment, width W may be set equal to a value between 0.4 fₛ and 0.5 fₛ responsive to the coefficient cff being smaller than 0.1. According to Condition (1b), in an exemplary embodiment, width W may be set equal to a value between 0.3 fₛ and 0.4 fₛ responsive to the coefficient cff being between 0.1 and 1.5. According to Condition (1c), in an exemplary embodiment, width W may be set equal to a value between 0.1 fₛ and 0.2 fₛ responsive to the coefficient cff being larger than 1.5. In an exemplary embodiment, the averaged J-point may be obtained by finding a maximum amplitude of averaged ECG signal 312 in a range of (tₛ, tₛ+W), where tₛ is a time instance corresponding to peak S′ of the S-wave. In an exemplary embodiment, point on averaged ECG signal 312 with a maximum amplitude in the selected range may be selected as the averaged J-point. Consequently, in an exemplary embodiment, the initial J-point may be replaced with the averaged J-point.

For further detail with respect to step 154, after obtaining the averaged J-point, an exemplary updated QRS complex may be extracted from averaged QRS complex 318 utilizing the averaged J-point detected location. In an exemplary embodiment, the updated QRS complex may include updated Q, R, and S edges which may be detected on averaged ECG signal 312 similar to detecting corresponding edges of averaged QRS complex 318, except that the initial J-point location may be replaced with the averaged J-point.

In further detail regarding step 156, in an exemplary embodiment, the number of edges in the updated QRS complex may be calculated by counting a number of slope changes in the updated QRS complex. In an exemplary embodiment, a derivative of the updated QRS complex may be obtained and a number of zero-crossings of the derivative may indicate the number of slope changes, and hence, the number of edges of the updated QRS complex.

For further detail with regards to step 158, in an exemplary embodiment, the temporal threshold may be set to 120 ms, which may be an upper limit for a narrow QRS complex. Therefore, an exemplary precondition for detecting an abnormal morphology in averaged ECG signal 312 may be an existence of narrow QRS complex in averaged ECG signal 312. In an exemplary embodiment, the lower limit for the number of edges of the updated QRS complex may be set to 3. In an exemplary embodiment, the lower limit for the number of edges may be empirically selected by examining different ECG signals associated with CAD. Therefore, in an exemplary embodiment, an abnormal morphology may be detected in an averaged ECG signal with a narrow averaged QRS complex that may have more than 3 edges.

In an exemplary embodiment, detecting the plurality of abnormal morphologies may include detecting the plurality of abnormal morphologies in at least 20% of the plurality of averaged ECG signals. In an exemplary embodiment, the plurality of averaged ECG signals may include a duration of at least 10 seconds. Therefore, in an exemplary embodiment, if at least 20% of the plurality of averaged ECG signals which have a total duration of at least 10 seconds include abnormal morphologies, raw ECG signal 302 may be determined to contain abnormal morphologies. FIG. 3F shows a plurality of averaged ECG signals 320 that include a plurality of abnormal morphologies 322, consistent with one or more exemplary embodiments of the present disclosure.

Referring again to FIG. 1A, in an exemplary embodiment, step 110 may include generating a plurality of fuzzy sets associated with the plurality of clinical data and the plurality of ECG features. An exemplary plurality of fuzzy sets may include a plurality of clinical symptoms fuzzy sets, a plurality of gender-age fuzzy sets, and a plurality of ECG fuzzy sets, an MI class, and a non-MI class.

In an exemplary embodiment, the plurality of clinical symptoms fuzzy sets may be associated with the plurality of clinical symptoms. An exemplary plurality of clinical symptoms fuzzy sets may include a typical MI fuzzy set, a high-risk for MI fuzzy set, an atypical MI fuzzy set, and a no MI symptom fuzzy set. In an exemplary embodiment, each of the first clinical symptom and the second clinical symptom (i.e., typical MI symptoms) may be mapped to a member of the typical MI fuzzy set through a corresponding membership function. In an exemplary embodiment, each of the third clinical symptom and the fourth clinical symptom (i.e., high-risk MI symptoms) may be mapped to a member of the high-risk for MI fuzzy set through a corresponding membership function. In an exemplary embodiment, the fifth clinical symptom (i.e., atypical MI symptoms) may be mapped to a member of the atypical MI fuzzy set through a corresponding membership function. In an exemplary embodiment, the sixth clinical symptom (i.e., non-MI symptoms or no symptoms at all) may be mapped to a member of the no MI symptom fuzzy set through a corresponding membership function.

In an exemplary embodiment, the plurality of gender-age fuzzy sets may be associated with the gender and the age of subject 210. An exemplary plurality of gender-age fuzzy sets may include a very low risk age for male fuzzy set, a very low risk age for female fuzzy set, a low risk age for male fuzzy set, a low risk age for female fuzzy set, a medium risk age for male fuzzy set, a medium risk age for female fuzzy set, a high risk age for male fuzzy set, a high risk age for female fuzzy set, a very high risk age for male fuzzy set, and a very high risk age for female fuzzy set. In an exemplary embodiment, a combination of an age lower than 30 years and a male gender may be mapped to a member of the very low risk age for male fuzzy set through a corresponding membership function. In an exemplary embodiment, a combination of an age lower than 40 years and a female gender may be mapped to a member of the very low risk age for female fuzzy set through a corresponding membership function. In an exemplary embodiment, a combination of an age between 30 and 40 years and a male gender may be mapped to a member of the low risk age for male fuzzy set through a corresponding membership function. In an exemplary embodiment, a combination of an age between 40 and 45 years and a female gender may be mapped to a member of the low risk age for female fuzzy set through a corresponding membership function. In an exemplary embodiment, a combination of an age between 40 and 50 years and a male gender may be mapped to a member of the medium risk age for male fuzzy set through a corresponding membership function. In an exemplary embodiment, a combination of an age between 45 and 50 years and a female gender may be mapped to a member of the medium risk age for female fuzzy set through a corresponding membership function. In an exemplary embodiment, a combination of an age between 50 and 55 and a male gender may be mapped to a member of the high risk age for male fuzzy set through a corresponding membership function. In an exemplary embodiment, a combination of an age between 50 and 55 years and a female gender may be mapped to a member of the high risk age for female fuzzy set through a corresponding membership function. In an exemplary embodiment, a combination of an age higher than 55 years and a male gender may be mapped to a member of the very high risk age for male fuzzy set through a corresponding membership function. In an exemplary embodiment, a combination of an age higher than 55 years and a female gender may be mapped to a member of the very high risk age for female fuzzy set through a corresponding membership function.

In an exemplary embodiment, the plurality of ECG fuzzy sets fuzzy sets may be associated with the plurality of ECG features. An exemplary plurality of ECG fuzzy sets may include an in favor of MI fuzzy set, a suspect of MI fuzzy set, and an apparently normal ECG fuzzy set. In an exemplary embodiment, each of the first plurality of features (i.e., in favor of MI ECG features) may be mapped to a member of the in favor of MI fuzzy set through a corresponding membership function. In an exemplary embodiment, each of the second plurality of features (i.e., suspect of MI ECG features) may be mapped to a member of the suspect of MI fuzzy set through a corresponding membership function. If none of an exemplary plurality of ECG features is detected in averaged ECG signal 312, it may be mapped to a member of the apparently normal ECG fuzzy set through a corresponding membership function.

Referring again to FIG. 1A, in an exemplary embodiment, step 111 may include designing a fuzzy inference system based on a set of rules. Each exemplary rule of the set of rules may include mapping a respective combination of a respective clinical symptoms fuzzy set of the plurality of clinical symptoms fuzzy sets, a respective gender-age fuzzy set of the plurality of gender-age fuzzy sets, and a respective ECG fuzzy set of the plurality of ECG fuzzy sets to one of the MI class or the non-MI class.

In an exemplary embodiment, there may be eighth combination of fuzzy sets that may be mapped to the MI class. An exemplary first combination may include the very high risk age for male fuzzy set and at least one of the suspect of MI fuzzy set, the in favor of MI fuzzy set, the typical MI fuzzy set, or the high-risk for MI fuzzy set. An exemplary second combination may include the high risk age for male fuzzy set and at least one of the following combinations: First, the in favor of MI fuzzy set and the atypical MI fuzzy set. Second, at least one of the typical MI fuzzy set or the high-risk for MI fuzzy set.

An exemplary third combination may include the medium risk age for male fuzzy set and at least one of the typical MI fuzzy set or the high-risk for MI fuzzy set. An exemplary fourth combination may include the low risk age for male fuzzy set and at least one of the following combinations: First, the in favor of MI fuzzy set and the no MI symptom fuzzy set. Second, at least one of the typical MI fuzzy set or the high-risk for MI fuzzy set.

An exemplary fifth combination may include the very low risk age for male fuzzy set and at least one of the following sub-combinations. First, the in favor of MI fuzzy set and at least one of the no MI symptom fuzzy set or the atypical MI fuzzy set. Second, at least one of the typical MI fuzzy set or the high-risk for MI fuzzy set.

An exemplary sixth combination may include the very high risk age for female fuzzy set and at least one of the in favor of MI fuzzy set, the typical MI fuzzy set, the high-risk for MI fuzzy set, or a combination of the suspect of MI fuzzy set and the no MI symptom fuzzy set. An exemplary seventh combination may include both of the following combinations: First, at least one of the high risk age for female fuzzy set, the medium risk age for female fuzzy set, or the low risk age for female fuzzy set. Second, at least one of the in favor of MI fuzzy set, the typical MI fuzzy set, or the high-risk for MI fuzzy set.

An exemplary eighth combination may include the very low risk age for female fuzzy set and at least one of the following combinations: First, the in favor of MI fuzzy set and the atypical MI fuzzy set. Second, at least one of the typical MI fuzzy set or the high-risk for MI fuzzy set.

In an exemplary embodiment, there may be a ninth combination that may include any combination of the above mentioned fuzzy sets that is different from each of the first combination, the second combination, the third combination, the fourth combination, the fifth combination, the sixth combination, the seventh combination, and the eighth combination. An exemplary ninth combination may be mapped to the non-MI class.

In an exemplary embodiment, the set of rules may include a total of 120 combinations of fuzzy sets that may be mapped to either the MI class or the non-MI class. Tables 1-10 show the mapping different combinations of fuzzy sets to the MI class or the non-MI class as described above

TABLE 1
Male_Age is Very_low_risk-age

| | ECG_Features | | |
|---|---|---|---|
| | Suspect_{—} | Apparently_{—} | In_favor_{—} |
| Clincal_symptoms | of_MI | Normal_ECG | of_MI |
| No_symptoms | Non-MI | Non-MI | MI |
| High_risk_for_MI | MI | MI | MI |
| Atypical_MI_symptoms | Non-MI | Non-MI | MI |
| Typical_MI_symptoms | MI | MI | MI |

TABLE 2
Male_Age is Low_risk_age

| | ECG_Features | | |
|---|---|---|---|
| | Suspect_{—} | Apparently_{—} | In_favor_{—} |
| Clincal_symptoms | of_MI | Normal_ECG | of_MI |
| No_symptoms | Non-MI | Non-MI | MI |
| High_risk_for_MI | MI | MI | MI |
| Atypical_MI_symptoms | Non-MI | Non-MI | MI |
| Typical_MI_symptoms | MI | MI | MI |

TABLE 3
Male_Age is Medium_risk_age

| | ECG_Features | | |
|---|---|---|---|
| | Suspect_{—} | Apparently_{—} | In_favor_{—} |
| Clincal_symptoms | of_MI | Normal_ECG | of_MI |
| No_symptoms | Non-MI | Non-MI | Non-MI |
| High_risk_for_MI | MI | MI | MI |
| Atypical_MI_symptoms | Non-MI | Non-MI | Non-MI |
| Typical_MI_symptoms | MI | MI | MI |

TABLE 4
Male_Age is High_risk_age

| | ECG_Features | | |
|---|---|---|---|
| | Suspect_{—} | Apparently_{—} | In_favor_{—} |
| Clincal_symptoms | of_MI | Normal_ECG | of_MI |
| No_symptoms | Non-MI | Non-MI | Non-MI |
| High_risk_for_MI | MI | MI | MI |
| Atypical_MI_symptoms | Non-MI | Non-MI | MI |
| Typical_MI_symptoms | MI | MI | MI |

TABLE 5
Male_Age is Very_high_risk_age

| | ECG_Features | | |
|---|---|---|---|
| | Suspect_{—} | Apparently_{—} | In_favor_{—} |
| Clincal_symptoms | of_MI | Normal_ECG | of_MI |
| No_symptoms | MI | Non-MI | MI |
| High_risk_for_MI | MI | MI | MI |
| Atypical_MI_symptoms | MI | Non-MI | MI |
| Typical_MI_symptoms | MI | MI | MI |

TABLE 6
Female_Age is Very_low_risk-age

| | ECG_Features | | |
|---|---|---|---|
| | Suspect_{—} | Apparently_{—} | In_favor_{—} |
| Clincal_symptoms | of_MI | Normal_ECG | of_MI |
| No_symptoms | Non-MI | Non-MI | Non-MI |
| High_risk_for_MI | MI | MI | MI |
| Atypical_MI_symptoms | Non-MI | Non-MI | MI |
| Typical_MI_symptoms | MI | MI | MI |

TABLE 7
Female_Age is Low_risk_age

| | ECG_Features | | |
|---|---|---|---|
| | Suspect_{—} | Apparently_{—} | In_favor_{—} |
| Clincal_symptoms | of_MI | Normal_ECG | of_MI |
| No_symptoms | Non-MI | Non-MI | MI |
| High_risk_for_MI | MI | MI | MI |
| Atypical_MI_symptoms | Non-MI | Non-MI | MI |
| Typical_MI_symptoms | MI | MI | MI |

TABLE 8
Female_Age is Medium_risk_age

| | ECG_Features | | |
|---|---|---|---|
| | Suspect_{—} | Apparently_{—} | In_favor_{—} |
| Clincal_symptoms | of_MI | Normal_ECG | of_MI |
| No_symptoms | Non-MI | Non-MI | MI |
| High_risk_for_MI | MI | MI | MI |
| Atypical_MI_symptoms | Non-MI | Non-MI | MI |
| Typical_MI_symptoms | MI | MI | MI |

TABLE 9
Female_Age is High_risk_age

| | ECG_Features | | |
|---|---|---|---|
| | Suspect_{—} | Apparently_{—} | In_favor_{—} |
| Clincal_symptoms | of_MI | Normal_ECG | of_MI |
| No_symptoms | Non-MI | Non-MI | MI |
| High_risk_for_MI | MI | MI | MI |
| Atypical_MI_symptoms | Non-MI | Non-MI | MI |
| Typical_MI_symptoms | M | MI | MI |

TABLE 10
Female_Age is Very_high_risk_age

| | ECG_Features | | |
|---|---|---|---|
| | Suspect_{—} | Apparently_{—} | In_favor_{—} |
| Clincal_symptoms | of_MI | Normal_ECG | of_MI |
| No_symptoms | MI | Non-MI | MI |
| High_risk_for_MI | MI | MI | MI |
| Atypical_MI_symptoms | Non-MI | Non-MI | MI |
| Typical_MI_symptoms | MI | MI | MI |

Referring again to FIG. 1A, in an exemplary embodiment, step 112 may include determining an occurrence of the heart attack in the subject utilizing the fuzzy inference system. FIG. 2B shows a schematic of a fuzzy inference system (FIS), consistent with one or more exemplary embodiments of the present disclosure. In an exemplary embodiment, processor 208 may be utilized to design an FIS 212. In an exemplary embodiment, FIS 212 may include a plurality of inputs, a fuzzifier 214, a fuzzy rule base 216, an inference engine 218, a defuzzifier 220, and an output Y. An exemplary plurality of inputs may include a first input X₁, a second input X₂, and a third input X₃.

In further detail with respect to step 112, in an exemplary embodiment, the plurality of clinical symptoms may be loaded to first input X₁. In an exemplary embodiment, the gender and the age of subject 210 may be loaded to second input X₂. In an exemplary embodiment, the plurality of ECG features may be mapped to third input X₃. In an exemplary embodiment, each of first input X₁, second input X₂, and third input X₃ may be mapped to a respective fuzzy input of a plurality of fuzzy inputs through a corresponding membership function. In an exemplary embodiment, first input X₁ may be mapped to a first fuzzy input μ₁. In an exemplary embodiment, first fuzzy input μ₁ may be a member of one or more of the plurality of clinical symptoms fuzzy sets. In an exemplary embodiment, second input X₂ may be mapped to a second fuzzy input μ₂. In an exemplary embodiment, second fuzzy input μ₂ may be a member of one or more of the plurality of clinical symptoms fuzzy sets. In an exemplary embodiment, third input X₃ may be mapped to a third fuzzy input μ₃. In an exemplary embodiment, third fuzzy input μ₃ may be a member of one or more of the plurality of ECG fuzzy sets.

In an exemplary embodiment, fuzzifier 214 may be configured to map first input X₁, second input X₂, and third input X₃ to first fuzzy input μ₁, second fuzzy input μ₂, and third fuzzy input μ₃, respectively, utilizing given formulas that assign more weights to inputs that are mapped to their corresponding fuzzy sets, as discussed above.

In an exemplary embodiment, inference engine 218 may be configured to map first fuzzy input μ₁, second fuzzy input μ₂, and third fuzzy input μ₃ to an inferred output μ_{Y} utilizing the set of rules described above. An exemplary set of rules may be stored in fuzzy rule base 216. In an exemplary embodiment, inferred output μ_{Y} may be associated with output Y. In an exemplary embodiment, defuzzifier 220 may be configured to map inferred output μ_{Y} to output Y. As a result, a fuzzy value of inferred output μ_{Y} may be mapped to a crisp value of output Y to determine whether the plurality of inputs belong to the MI class or the non-MI class.

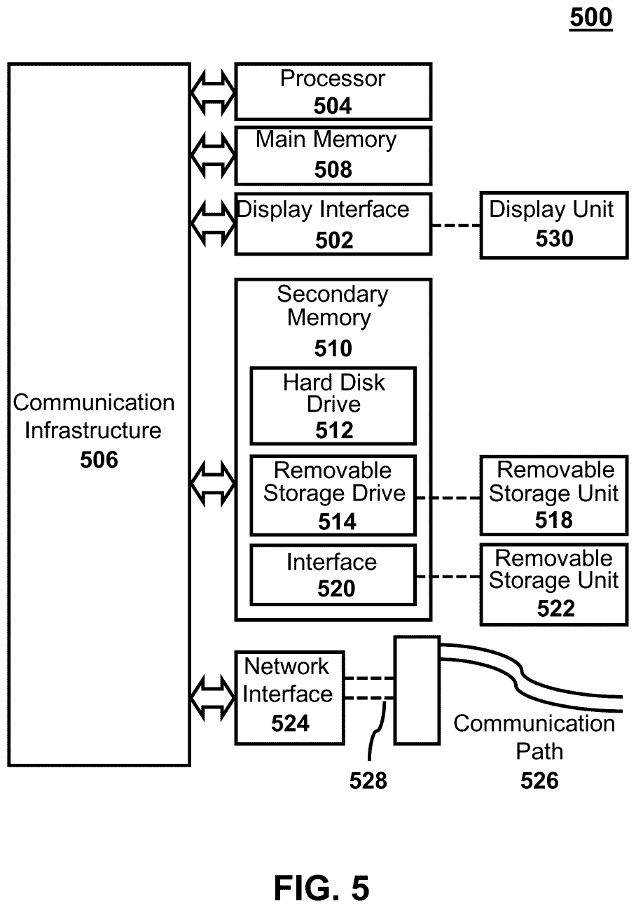

FIG. 5 shows an example computer system 500 in which an embodiment of the present invention, or portions thereof, may be implemented as computer-readable code, consistent with exemplary embodiments of the present disclosure. For example, method 100 may be implemented in computer system 500 using hardware, software, firmware, tangible computer readable media having instructions stored thereon, or a combination thereof and may be implemented in one or more computer systems or other processing systems. Hardware, software, or any combination of such may embody any of the modules and components in FIGS. 1A-2B.

If programmable logic is used, such logic may execute on a commercially available processing platform or a special purpose device. One ordinary skill in the art may appreciate that an embodiment of the disclosed subject matter can be practiced with various computer system configurations, including multi-core multiprocessor systems, minicomputers, mainframe computers, computers linked or clustered with distributed functions, as well as pervasive or miniature computers that may be embedded into virtually any device.

For instance, a computing device having at least one processor device and a memory may be used to implement the above-described embodiments. A processor device may be a single processor, a plurality of processors, or combinations thereof. Processor devices may have one or more processor “cores.”

An embodiment of the invention is described in terms of this example computer system 500. After reading this description, it will become apparent to a person skilled in the relevant art how to implement the invention using other computer systems and/or computer architectures. Although operations may be described as a sequential process, some of the operations may in fact be performed in parallel, concurrently, and/or in a distributed environment, and with program code stored locally or remotely for access by single or multi-processor machines. In addition, in some embodiments the order of operations may be rearranged without departing from the spirit of the disclosed subject matter.

Processor device 504 may be a special purpose or a general-purpose processor device. As will be appreciated by persons skilled in the relevant art, processor device 504 may also be a single processor in a multi-core/multiprocessor system, such system operating alone, or in a cluster of computing devices operating in a cluster or server farm. Processor device 504 may be connected to a communication infrastructure 506, for example, a bus, message queue, network, or multi-core message-passing scheme.

In an exemplary embodiment, computer system 500 may include a display interface 502, for example a video connector, to transfer data to a display unit 530, for example, a monitor. Computer system 500 may also include a main memory 508, for example, random access memory (RAM), and may also include a secondary memory 510. Secondary memory 510 may include, for example, a hard disk drive 512, and a removable storage drive 514. Removable storage drive 514 may include a floppy disk drive, a magnetic tape drive, an optical disk drive, a flash memory, or the like. Removable storage drive 514 may read from and/or write to a removable storage unit 518 in a well-known manner. Removable storage unit 518 may include a floppy disk, a magnetic tape, an optical disk, etc., which may be read by and written to by removable storage drive 514. As will be appreciated by persons skilled in the relevant art, removable storage unit 518 may include a computer usable storage medium having stored therein computer software and/or data.

In alternative implementations, secondary memory 510 may include other similar means for allowing computer programs or other instructions to be loaded into computer system 500. Such means may include, for example, a removable storage unit 522 and an interface 520. Examples of such means may include a program cartridge and cartridge interface (such as that found in video game devices), a removable memory chip (such as an EPROM, or PROM) and associated socket, and other removable storage units 522 and interfaces 520 which allow software and data to be transferred from removable storage unit 522 to computer system 500.

Computer system 500 may also include a communications interface 524. Communications interface 524 allows software and data to be transferred between computer system 500 and external devices. Communications interface 524 may include a modem, a network interface (such as an Ethernet card), a communications port, a PCMCIA slot and card, or the like. Software and data transferred via communications interface 524 may be in the form of signals, which may be electronic, electromagnetic, optical, or other signals capable of being received by communications interface 524. These signals may be provided to communications interface 524 via a communications path 526. Communications path 526 carries signals and may be implemented using wire or cable, fiber optics, a phone line, a cellular phone link, an RF link or other communications channels.

In this document, the terms “computer program medium” and “computer usable medium” are used to generally refer to media such as removable storage unit 518, removable storage unit 522, and a hard disk installed in hard disk drive 512. Computer program medium and computer usable medium may also refer to memories, such as main memory 508 and secondary memory 510, which may be memory semiconductors (e.g. DRAMs, etc.).

Computer programs (also called computer control logic) are stored in main memory 508 and/or secondary memory 510. Computer programs may also be received via communications interface 524. Such computer programs, when executed, enable computer system 500 to implement different embodiments of the present disclosure as discussed herein. In particular, the computer programs, when executed, enable processor device 504 to implement the processes of the present disclosure, such as the operations in method 100 illustrated by flowcharts of FIGS. 1A-1E discussed above. Accordingly, such computer programs represent controllers of computer system 500. Where an exemplary embodiment of method 100 is implemented using software, the software may be stored in a computer program product and loaded into computer system 500 using removable storage drive 514, interface 520, and hard disk drive 512, or communications interface 524.

Embodiments of the present disclosure also may be directed to computer program products including software stored on any computer useable medium. Such software, when executed in one or more data processing device, causes a data processing device to operate as described herein. An embodiment of the present disclosure may employ any computer useable or readable medium. Examples of computer useable mediums include, but are not limited to, primary storage devices (e.g., any type of random access memory), secondary storage devices (e.g., hard drives, floppy disks, CD ROMS, ZIP disks, tapes, magnetic storage devices, and optical storage devices, MEMS, nanotechnological storage device, etc.).

The embodiments have been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

### EXAMPLE

In this example, an implementation of method 100 for early, non-invasive and fast detection of heart attack is demonstrated. Three specific datasets were collected from different cohorts of patients containing a total of 620 patients. The first cohort of patients consists of 500 patients from hospital #1 (Cohort #1), 54 patients from hospital #2 (Cohort #2), and 66 patients from hospital #3 (Cohort #2). Also, 500 individuals were selected from some apparently healthy volunteers as the control (Healthy) group.

Generally, cohort #1 known as the retrospective evaluation population for the present method includes a group of individuals who had been referred to either the emergency department (ED) or the cardiac care unit (CCU) of hospital #1 and had been diagnosed as an MI (heart attack) candidate by the medical experts there. As a retrospective study, their ECG at the time of admission along with a list of clinical symptoms, age, gender, and other health data at that time of presenting in the hospital had been collected to be used for the evaluation of an implementation of method 100. Digitalized 12-lead ECG signals for a duration of approximately 10 seconds with a 12-lead standard ECG unit were obtained before undergoing either coronary angiography or any specific medical assessments and treatments. For cohort #1, a pair of coronary angiography reports and troponin level testing had been defined as the gold standard.

Cohort #2 consists of a total of 54 individuals hospitalized at hospital #2 and waiting for undergoing angiography. It was a prospective observational study including consecutive patients hospitalized at the CCU or electively addressed to the coronary angiography laboratory for coronary angiography examination. Using a standard 12-lead ECG machine, a digitalized long-term single-lead ECG for about 4 minutes along with a short-term standard 12-lead ECG (for a duration of approximately 10 seconds) was collected from each MI patient. Additionally, after initial medical examinations, a set of clinical signs and symptoms associated with their disease were collected at the time of their presentation to the hospital.

Cohort #3 consists of a total of 66 individuals referring to the ED or CCU of hospital #3 complaining about some unexpected clinical symptoms, such as pain and discomfort in some specific parts of their body associated with some other sign and symptoms such as sudden fainting, sweating, shortness of breath, vomiting, nausea light headedness with or without some past history of risk factors such as the history of diabetes, hypertension, and hyperlipidemia. According to the health status at the admission time, these individuals were not in a significant unstable emergency situation with an acute condition, however, since they were alert to the signs of a possible heart attack, they had been recommended to get followed. After initial medical examinations by the cardiologists and other CCU experts, measurement of the troponin level by a blood testing was prescribed for most of them.

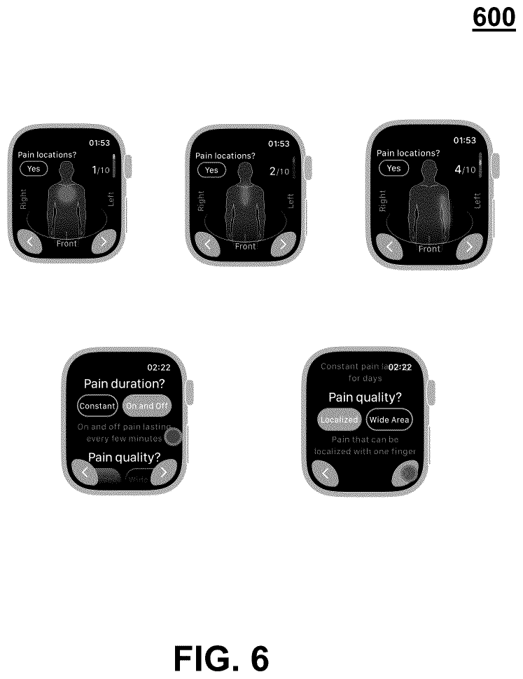

FIG. 6 shows different pages of a software application for early detection of heart attack implemented on a smart watch, consistent with one or more exemplary embodiments of the present disclosure. During the waiting time to receive the troponin result, which routinely takes some time, without inducing a delay in any emergency care and treatment, the patients were asked to wear an ECG-based smartwatch and work with an implemented software 600 on the smartwatch. A subject recorded a 30-second lead I (right arm [−] to left arm [+]) ECG on the watch by pressing the crown with a finger of the hand opposite the hand with the watch body electrode. Afterwards, the clinical symptoms were directly acquired by the subject using the software implemented on the smartwatch. Finally, an implementation of method 100 was applied to the obtained data and the MI or non_MI cases were detected.

Despite the availability of more than one lead ECG from cohorts #1 and #2, to have a meaningful comparison with the analysis result of cohort #3, only one lead of ECG (lead I) was used for analysis by the method. For the aforementioned cohort of patients, the single-lead ECGs (only Lead I) as well as the clinical symptoms features were analyzed together and then evaluated for the detection of MI by a rule-based fuzzy inference, blinded to the angiographic or troponin testing results.

For each individual, a vector containing only zero or one value was generated describing the feeling or not feeling of the clinical sign and symptoms of upper chest pain, middle chest pain, upper abdomen pain, pain in the neck, pain in the jaw, pain in the right shoulder, pain in the left shoulder, pain inside the right arm, pain inside the left arm, pain between shoulders in back, fainting, sweating, shortness of breath, light headedness, vomiting, nausea, history of diabetes, history of hypertension, and history of hyperlipidemia. In the next step, to map the vector into one number, a number of pre-defined weight numbers were multiplied by each value and consequently, the sum of the produced values was generated to show the mapped number. A resultant number was then considered as the input value representing the clinical symptoms of the patient and its membership value to each of the designed membership functions of the clinical symptoms fuzzy set was calculated.

To analyze the ECG, after a multi-layer preprocessing, filter bank implementations and strict and precise wave delineation of averaged ECG, an implementation of method 100 looked for the existence or non-existence of ST segment elevation (elevation of the ST segment compared to the isoelectric line), ST segment depression (depression of the ST segment compared to the isoelectric line), deformation and angulation of the ST segment, pathological changed Q-wave, morphological changed QRS complex, tall T-wave, inverted T-wave, tented T-wave, flattened T-wave, biphasic T-wave, and severe bradycardia.

If the abnormal morphology, such as notching or slurring shapes in the QRS complex was detected in averaged ECGs and the percentage of the occurrence of such abnormality was more than 20% of the entire ECG averages then the value of the vector of ECG features was 1 and otherwise is zero.

If the pathological changed Q-wave was detected in averaged ECGs and the percentage of the occurrence of such abnormality was more than 20% of the entire ECG averages, then the value of the vector of ECG features was 1 and otherwise is zero.

If each of the ST segment elevation or ST segment depression or deformation of the ST segment or tall T-wave or inverted T-wave or tented T-wave or flattened T-wave or biphasic T-wave was detected in averaged ECGs and the percentage of the occurrence of such feature was more than 70% of the entire ECG averages, then the value of the vector of ECG features was 1 and otherwise was zero. The existence or non-existence of bradycardia was either shown by zero or one. Therefore, for each individual, a vector containing only zero or one value was generated describing the existence or non-existence of the above-mentioned ECG criteria. In the next step, to map such vector into one number, the pre-defined weight numbers were multiplied by each value and consequently, the sum of the produced values was generated to show the mapped number. The resulting number was then considered as the input value representing the ECG features of the patient and its membership value to each of the designed membership functions of the ECG features fuzzy set was calculated. According to the specific designed age-gender fuzzy set, the fuzzy inputs for each individual's age and its membership value to each of the designed membership functions were then calculated. Using such fuzzy inputs and an implementation of FIS 212, the fuzzy inputs were applied to the fuzzy system and finally, the output representing the fuzzy membership value of that input to output membership functions (Non-MI case or MI cases) was calculated using the “Mamdani” inference method for fuzzy systems.

The evaluation of the disclosed method with the available cohort of patients was performed according to the comparison with the available single-lead ECG (Lead I) from the control group. For the cohort #1, an accuracy of about 91.3% and 88.2% was achieved for sensitivity and positive predictive values, respectively. Also, for cohort #2, an accuracy of about 96.29% and 98.11% was achieved for sensitivity and positive predictive values, respectively. For the cohort #3, an accuracy of about 96.7% and 92.2% was achieved for sensitivity and positive predictive values, respectively.

While the foregoing has described what may be considered to be the best mode and/or other examples, it is understood that various modifications may be made therein and that the subject matter disclosed herein may be implemented in various forms and examples, and that the teachings may be applied in numerous applications, only some of which have been described herein. It is intended by the following claims to claim any and all applications, modifications and variations that fall within the true scope of the present teachings.

Unless otherwise stated, all measurements, values, ratings, positions, magnitudes, sizes, and other specifications that are set forth in this specification, including in the claims that follow, are approximate, not exact. They are intended to have a reasonable range that is consistent with the functions to which they relate and with what is customary in the art to which they pertain.

The scope of protection is limited solely by the claims that now follow. That scope is intended and should be interpreted to be as broad as is consistent with the ordinary meaning of the language that is used in the claims when interpreted in light of this specification and the prosecution history that follows and to encompass all structural and functional equivalents.

Except as stated immediately above, nothing that has been stated or illustrated is intended or should be interpreted to cause a dedication of any component, step, feature, object, benefit, advantage, or equivalent to the public, regardless of whether it is or is not recited in the claims.

It will be understood that the terms and expressions used herein have the ordinary meaning as is accorded to such terms and expressions with respect to their corresponding respective areas of inquiry and study except where specific meanings have otherwise been set forth herein. Relational terms such as first and second and the like may be used solely to distinguish one entity or action from another without necessarily requiring or implying any actual such relationship or order between such entities or actions. The terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus. An element proceeded by “a” or “an” does not, without further constraints, preclude the existence of additional identical elements in the process, method, article, or apparatus that comprises the element.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various implementations. This is for purposes of streamlining the disclosure, and is not to be interpreted as reflecting an intention that the claimed implementations require more features than are expressly recited in each claim. Rather, as the following claims reflect, inventive subject matter lies in less than all features of a single disclosed implementation. Thus, the following claims are hereby incorporated into the Detailed Description, with each claim standing on its own as a separately claimed subject matter. While various implementations have been described, the description is intended to be exemplary, rather than limiting and it will be apparent to those of ordinary skill in the art that many more implementations and implementations are possible that are within the scope of the implementations. Although many possible combinations of features are shown in the accompanying figures and discussed in this detailed description, many other combinations of the disclosed features are possible. Any feature of any implementation may be used in combination with or substituted for any other feature or element in any other implementation unless specifically restricted. Therefore, it will be understood that any of the features shown and/or discussed in the present disclosure may be implemented together in any suitable combination. Accordingly, the implementations are not to be restricted except in light of the attached claims and their equivalents. Also, various modifications and changes may be made within the scope of the attached claims.

1. A method for early detection of a heart attack in a subject, the method comprising:
acquiring a plurality of clinical symptoms from the subject;
acquiring a gender of the subject, the gender comprising one of a male or a female;
acquiring an age of the subject;
acquiring a raw electrocardiography (ECG) signal from the subject at a diagnosis time period;
generating, utilizing one or more processors, a denoised ECG signal by applying a first wavelet transform on the raw ECG signal;
generating, utilizing the one or more processors, an artifact-free ECG signal by applying a second wavelet transform on the denoised ECG signal;
generating, utilizing the one or more processors, a filtered ECG signal by applying a finite impulse response (FIR) filter on the artifact-free ECG signal;
extracting, utilizing the one or more processors, an averaged ECG signal from the filtered ECG signal, the averaged ECG signal comprising a QRS complex, an ST segment, and a T wave;
acquiring a plurality of ECG features from the averaged ECG signal and the filtered ECG signal;
generating, utilizing the one or more processors, a plurality of clinical symptoms fuzzy sets associated with the plurality of clinical symptoms;
generating, utilizing the one or more processors, a plurality of gender-age fuzzy sets associated with the gender and the age;
generating, utilizing the one or more processors, a plurality of ECG fuzzy sets associated with the plurality of ECG features;
generating, utilizing the one or more processors, a myocardial infarction (MI) class corresponding to occurrence of an MI in the subject and a non-MI class corresponding to an absence of MI in the subject;
designing, utilizing the one or more processors, a fuzzy inference system based on a set of rules, each rule of the set of rules comprising mapping a respective combination of a respective clinical symptoms fuzzy set of the plurality of clinical symptoms fuzzy sets, a respective gender-age fuzzy set of the plurality of gender-age fuzzy sets, and a respective ECG fuzzy set of the plurality of ECG fuzzy sets to one of the MI class or the non-MI class;
mapping each of the plurality of clinical symptoms, the gender, the age, and the plurality of ECG features to a respective fuzzy input of a plurality of fuzzy inputs;
determining, utilizing the fuzzy inference system, an occurrence of the heart attack in the subject by applying the plurality of fuzzy inputs to the fuzzy inference system.

2. The method of claim 1, wherein acquiring the plurality of clinical symptoms comprises assessment of:
a first clinical symptom comprising:
on/off pain with a continuous duration of at least five minutes during a one hour period before the diagnosis time in at least one of a first plurality of regions having a total size larger than three times of a size of a fingertip of the subject, the first plurality of regions comprising upper chest, middle chest (sternum), upper abdomen, neck, jaw, right shoulder, left shoulder, inside right arm, inside left arm, and between shoulders in back;
a second clinical symptom comprising:
during a one hour period before the diagnosis time, at least one of fainting or all of:
at least one of shortness of breath, light headedness, diabetes, and hypertension; and
at least one of sweating and on/off pain with a continuous duration of at least five minutes;
a third clinical symptom comprising:
on/off pain with a continuous duration of at least five minutes from 24 hours until one hour before the diagnosis time in at least one of the first plurality of regions;
a fourth clinical symptom comprising:
from 24 hours until one hour before the diagnosis time, fainting or all of:
at least one of shortness of breath, light headedness, diabetes, and hypertension; and
at least one of sweating and on/off pain with a continuous duration of at least five minutes;
a fifth clinical symptom comprising:
atypical MI pain during a one hour period before the diagnosis time in at least one of a second plurality of regions comprising upper chest, middle chest (sternum), upper abdomen, neck, jaw, right shoulder, left shoulder, inside right arm, inside left arm, and between shoulders in back, the atypical MI pain being continuous or in an area smaller than a size of the fingertip; and
a sixth clinical symptom different from each of the first clinical symptom, the second clinical symptom, the third clinical symptom, the fourth clinical symptom, and the fifth clinical symptom.

3. The method of claim 2, wherein acquiring the plurality of ECG features comprises:
assessment of a first plurality of features in the averaged ECG signal, the first plurality of features comprising:
an elevation or a depression in the ST segment;
a pathologic Q-wave or an abnormal morphology in the QRS complex; and
the T wave comprising a tall T wave; and
assessment of a second plurality of features in one of the averaged ECG signal and the filtered ECG signal, the second plurality of features comprising:
a deformation in the ST segment;
a severe bradycardia in the filtered ECG signal; and
the T wave comprising an inverted T wave, a tent T wave, a flat T wave, or a biphasic T wave.

4. The method of claim 3, wherein generating the plurality of clinical symptoms fuzzy sets comprises generating:
a typical MI fuzzy set associated with at least one of the first clinical symptom and the second clinical symptom;
a high-risk for MI fuzzy set associated with at least one of the third clinical symptom and the fourth clinical symptom;
an atypical MI fuzzy set associated with the fifth clinical symptom; and
a no MI symptom fuzzy set associated with the sixth clinical symptom.

5. The method of claim 4, wherein generating the plurality of gender-age fuzzy sets comprises generating:
a very low risk age for male fuzzy set associated with the age being lower than 30 years and the gender being male;
a very low risk age for female fuzzy set associated with the age being lower than 40 years and the gender being female;
a low risk age for male fuzzy set associated with the age being between 30 and 40 years and the gender being male;
a low risk age for female fuzzy set associated with the age being between 40 and 45 years and the gender being female;
a medium risk age for male fuzzy set associated with the age being between 40 and 50 years and the gender being male;
a medium risk age for female fuzzy set associated with the age being between 45 and 50 years and the gender being female;
a high risk age for male fuzzy set associated with the age being between 50 and 55 years and the gender being male;
a high risk age for female fuzzy set associated with the age being between 50 and 55 years and the gender being female;
a very high risk age for male fuzzy set associated with the age being higher than 55 years and the gender being male; and
a very high risk age for female fuzzy set associated with the age being higher than 55 years and the gender being female.

6. The method of claim 5, wherein generating the plurality of ECG fuzzy sets comprises generating:
an in favor of MI fuzzy set associated with at least one of the first plurality of features;
a suspect of MI fuzzy set associated with at least one of the second plurality of features; and
an apparently normal ECG fuzzy set.

7. The method of claim 6, wherein mapping the respective combination to the one of the MI class or the non-MI class comprises mapping a first combination to the MI class, the first combination comprising:
the very high risk age for male fuzzy set; and
at least one of the suspect of MI fuzzy set, the in favor of MI fuzzy set, the typical MI fuzzy set, or the high-risk for MI fuzzy set.

8. The method of claim 7, wherein mapping the respective combination to the one of the MI class or the non-MI class further comprises mapping a second combination to the MI class, the second combination comprising:
the high risk age for male fuzzy set; and
at least one of:
the in favor of MI fuzzy set and the atypical MI fuzzy set; or
at least one of the typical MI fuzzy set or the high-risk for MI fuzzy set.

9. The method of claim 8, wherein mapping the respective combination to the one of the MI class or the non-MI class further comprises mapping a third combination to the MI class, the third combination comprising:
the medium risk age for male fuzzy set; and
at least one of the typical MI fuzzy set or the high-risk for MI fuzzy set.

10. The method of claim 9, wherein mapping the respective combination to the one of the MI class or the non-MI class further comprises mapping a fourth combination to the MI class, the fourth combination comprising:
the low risk age for male fuzzy set; and
at least one of:
the in favor of MI fuzzy set and the no MI symptom fuzzy set; or
at least one of the typical MI fuzzy set or the high-risk for MI fuzzy set.

11. The method of claim 10, wherein mapping the respective combination to the one of the MI class or the non-MI class further comprises mapping a fifth combination to the MI class, the fifth combination comprising:
the very low risk age for male fuzzy set; and
at least one of:
the in favor of MI fuzzy set and at least one of the no MI symptom fuzzy set or the atypical MI fuzzy set; or
at least one of the typical MI fuzzy set or the high-risk for MI fuzzy set.

12. The method of claim 11, wherein mapping the respective combination to the one of the MI class or the non-MI class further comprises mapping a sixth combination to the MI class, the sixth combination comprising:
the very high risk age for female fuzzy set; and
at least one of:
the in favor of MI fuzzy set;
the typical MI fuzzy set;
the high-risk for MI fuzzy set; or
the suspect of MI fuzzy set and the no MI symptom fuzzy set.

13. The method of claim 12, wherein mapping the respective combination to the one of the MI class or the non-MI class further comprises mapping a seventh combination to the MI class, the seventh combination comprising:
at least one of the high risk age for female fuzzy set, the medium risk age for female fuzzy set, or the low risk age for female fuzzy set; and
at least one of the in favor of MI fuzzy set, the typical MI fuzzy set, or the high-risk for MI fuzzy set.

14. The method of claim 13, wherein mapping the respective combination to the one of the MI class or the non-MI class further comprises mapping an eighth combination to the MI class, the eighth combination comprising:
the very low risk age for female fuzzy set; and
at least one of:
the in favor of MI fuzzy set and the atypical MI fuzzy set; or
at least one of the typical MI fuzzy set or the high-risk for MI fuzzy set.

15. The method of claim 14, wherein mapping the respective combination to the one of the MI class or the non-MI class further comprises mapping a ninth combination to the non-MI class, the ninth combination different from each of the first combination, the second combination, the third combination, the fourth combination, the fifth combination, the sixth combination, the seventh combination, and the eighth combination.

16. The method of claim 15, wherein mapping each of the plurality of clinical symptoms, the gender, the age, and the plurality of ECG features to a respective fuzzy input of a plurality of fuzzy inputs comprises:
mapping the plurality of clinical symptoms to a first fuzzy input of the plurality of fuzzy inputs, the first fuzzy input associated with the plurality of clinical symptoms fuzzy sets;
mapping the gender and the age to a second fuzzy input of the plurality of fuzzy inputs, the second fuzzy input associated with the plurality of gender-age fuzzy sets; and
mapping the plurality of ECG features to a third fuzzy input of the plurality of fuzzy inputs, the third fuzzy input associated with the plurality of ECG fuzzy sets.
